(19) United States
(12) Patent Application Publication
(10) Pub. No.: US 2023/0046382 A1
(43) Pub. Date: Feb. 16, 2023

(54) INCONTINENCE ARTICLE WITH PH REGULATOR

(71) Applicant: PAUL HARTMANN AG, Heidenheim Baden-Württemberg (DE)

(72) Inventors: Christian BÄHRLE, Herbrechtingen (DE); Juan ESQUERRA, Montornès del Vallés, Barcelona (ES); Enno GAUSE, Heidenheim Baden-Württemberg (DE); Luis HIDALGO, Montornès del Vallés, Barcelona (ES); Rüdiger KESSELMEIER, Burlafingen (DE); Isabel STALTER, Blaustein (DE); Maximilian SWEREV, Augsburg Bayern (DE)

(21) Appl. No.: 17/786,652

(22) PCT Filed: Dec. 18, 2020
(86) PCT No.: PCT/EP2020/086915

§ 371 Date: Jun. 17, 2022

(30) Foreign Application Priority Data
Dec. 20, 2019 (EP) 19218866.2

(51) Int. Cl. A61F 13/84; A61L 15/20; A61L 15/46

(52) U.S. Cl. CPC A61F 13/8405; A61L 15/20; A61L 15/46; A61F 2013/530481

(57) ABSTRACT

The present invention relates to an incontinence article for absorption of bodily excretions, comprising an at least regionally liquid-permeable topsheet, a substantially liquid-impermeable backsheet, and an absorption body arranged between the topsheet and the backsheet, the incontinence article comprising a pH regulator between the topsheet and the backsheet at least regionally, characterized in that the pH regulator comprises monosodium citrate or disodium citrate.

The present invention relates to an incontinence article for absorption of bodily excretions, comprising an agent for regulation of pH, to be preferably used by adults, and to arrangements of such incontinence articles.

A common problem with continuous use of incontinence articles is the occurrence of skin irritation or dermatitis caused by prolonged contact of the skin with bodily excretions such as, for example, urine. So-called incontinence-associated dermatitis (IAD), an inflammation of the skin in the perineal or perigenital region that is caused by contact with an irritant such as urine, commonly occurs in incontinent elderly people in particular. A major role is played here by changes in the pH of the skin upon contact with the more alkaline urine. The frequent occurrence of IAD in elderly people correlates with an age-related weakened skin barrier and with a reduced capacity of the skin for regeneration. Moreover, people who suffer from incontinence for medical reasons or because of age frequently rely on the support of nursing staff. Especially at night, regular checking of the incontinence article used with a view to a prompt change after a micturition event is prevented by the need of the individual concerned for undisturbed sleep. Therefore, it is precisely here that the skin often comes into prolonged contact with bodily excretions. In the case of individuals for whom frequent micturition events occur at short intervals, it would be advantageous for improving the quality of life and for cost reasons if the incontinence articles did not have to be promptly changed after every micturition event.

Therefore, there is a need for incontinence articles having improved skin compatibility, especially when there is a high frequency of micturition or relatively long intervals between changes.

Incontinence articles for adults have been known for a long time and commonly comprise an at least regionally liquid-permeable topsheet, a substantially liquid-impermeable backsheet, and an absorption body arranged between the topsheet and the backsheet. In principle, they can be designed in a variety of ways, for example as incontinence products in direct contact with the body or as absorbent bed pads.

In the prior art, there are approaches to provide incontinence articles having an agent for pH control. WO2012121932A1 teaches the use of a buffer solution comprising a weak acid or a weak base and the respective corresponding salt, for example citric acid and sodium citrate in a specific mixing ratio. In general technical language, the term sodium citrate refers to the tribasic sodium salt of citric acid, also known as trisodium citrate.

The inventors have discovered that the use of a buffer mixture composed of citric acid and sodium citrate has the disadvantage that the production of a homogeneous mixture is technically complicated and at least partial demixing and nonuniform distribution of the individual components can easily occur in the production process. As a result, the input of the components into the incontinence articles, which are usually produced in high-speed production machines, may be erroneous and may not be specification-compliant. This is associated with the risk that the quantity and/or the distribution of the components of the buffer mixture—and thus the desired buffer effect being ensured upon intended use of the articles—is subject to strong variations between individual incontinence articles or within an incontinence article. The risk of local accumulation of citric acid means that the pH of the incontinence article in that region is highly acidic, i.e., the pH in that region falls distinctly below a level beneficial to skin health.

It is therefore an object of the present invention to overcome these disadvantages.

It is a further object of the invention to provide users who have different needs with incontinence articles having improved skin-friendliness.

This object is achieved by an incontinence article for absorption of bodily excretions, comprising an at least regionally liquid-permeable topsheet, a substantially liquid-impermeable backsheet, and an absorption body arranged between the topsheet and the backsheet, the incontinence article comprising a pH regulator between the topsheet and the backsheet at least regionally, characterized in that the pH regulator comprises monosodium citrate or disodium citrate. Thus, the pH regulator may preferably comprise either monosodium citrate or disodium citrate, or monosodium citrate and disodium citrate.

The incontinence article according to the invention can achieve a relatively uniform pH over multiple micturition events. A further advantage when using monosodium citrate is, for example, that monosodium citrate is less hygroscopic than, for example, citric acid, meaning that undesired entry of moisture into the incontinence article before use can be avoided.

Since monosodium citrate is less hygroscopic than, for example, citric acid, a further advantage is better pourability, meaning that the metered addition of monosodium citrate can be done more easily.

The backsheet can be formed, in particular, by a breathable, but substantially liquid-tight, film material. The topsheet is preferably formed, at least regionally, from a nonwoven-based, at least regionally liquid-permeable material.

In a preferred embodiment, the pH regulator additionally comprises trisodium citrate, meaning that the pH regulator may preferably comprise monosodium citrate and trisodium citrate, or disodium citrate and trisodium citrate, or monosodium citrate and disodium citrate and trisodium citrate. As a result, the buffering effect of an incontinence article can be fine-tuned depending on what is striven for as regards the usage period or absorption capacity of the article, without the risk of development of highly acidic regions, as is the case when using citric acid.

In such a case, the pH regulator preferably comprises a homogeneous mixture of the respective citrates. This has the advantage that local differences in the buffering effect of the pH regulator can be avoided.

The terms monosodium citrate, disodium citrate and trisodium citrate encompass both the nonhydrogenated form and hydrates of the respective substance.

In a preferred embodiment, the pH regulator consists of monosodium citrate.

In a further preferred embodiment, the pH regulator consists of disodium citrate.

The use of only one component as pH regulator has technical advantages, since it is not necessary to mix various components or to maintain homogeneous mixing during the production process. Moreover, a uniform buffering effect across the regions of the incontinence article that contain pH regulator is further supported.

It has been found to be advantageous if the pH regulator is particulate.

Preferably, at least 50 percent by weight of the pH regulator has a particle size of 10 to 2000 μm, in particular of 50 to 1200 μm, further in particular of 80 to 800 μm.

In principle, pH regulators having a smaller or larger particle size are also available and usable.

However, the preferred particle sizes offer the advantage that particles of such a size dissolve more slowly, when wetted with bodily excretions such as a micturition fluid, than pH regulators of a smaller particle size, meaning that the pH regulator is not washed out as rapidly during use and a pH-regulating effect is preserved for longer on a topside of the incontinence article that faces the user's skin.

Moreover, a pH regulator of the preferred particle size can undergo metered addition and be incorporated into the incontinence article more easily than, for example, finely powdered pH regulators, particularly in high-speed machines, since there is less dust formation and associated material loss in the production process.

On the other hand, particles of this size also offer the advantage that they are haptically less perceptible during use of the incontinence article than, for example, granules of a larger particle size, thereby improving the wearing comfort of the incontinence article.

As an alternative to a particulate pH regulator, the pH regulator can also be introduced in dissolved form, in particular as an aqueous or alcoholic solution, into the incontinence article, in particular by spraying or immersion and optionally subsequent drying of at least one absorption body layer.

As a further alternative, the incontinence article can contain an additional material, in particular a planar material, pretreated with the pH regulator, in particular a nonwoven material or cellulose material. Preferably, such a planar material can be arranged between the liquid absorption layer and a storage layer of the absorption body.

In a preferred embodiment, the amount of pH regulator is 10 to 100 g/m², in particular 20 to 80 g/m², further in particular 25 to 60 g/m². The amount of pH regulator is based on the surface area of the pH regulator-covered region of the incontinence article spread out flat. The above-specified amount of pH regulator is to be understood to mean that it is based on an anhydrous pH regulator, for example nonhydrogenated monosodium citrate. If the pH regulator comprises one or more hydrates or, as described below, more than 5%, in particular more than 10%, additive substances, the total amount of pH regulator in g/m² is adjusted in such a way that the total amount of the pure and/or or nonhydrogenated pH regulator present corresponds to the preferred amount above.

Preferably, the monosodium citrate or disodium citrate or trisodium citrate is used substantially in pure form, in particular with a degree of purity of at least 90%, further in particular at least 95%, further in particular at least 98%, further in particular at least 99%, further in particular 100%. As a result, skin irritation caused by residues of other substances when using the incontinence article can be substantially avoided.

However, it is also conceivable that the pH regulator comprises a small amount of one or more additive substances, for example packaging and/or conditioning agents such as release agents, stabilizers, dust-binding agents, anticaking agents, wetting agents, coagulants, anticoagulants, adhesive or surface-active substances or antimicrobial substances, dyes or fragrances, or pH indicators. In such a case, the proportion of the one or more other substances, based on the total amount of pH regulator, is altogether preferably less than 10%, further preferably less than 5%, further preferably less than 2%, further preferably less than 1%.

The absorption body of the incontinence article is suited and intended for absorbing and permanently storing bodily excretions, in particular body fluids, in particular urine. To this end, the absorption body advantageously comprises at least one storage layer which preferably comprises a superabsorbent polymer (SAP).

Alternatively, the absorption body can also comprise more than one, in particular at least two, storage layers.

The absorption body can comprise one or more channels in one or more than one storage layer.

The absorption body can be rectangular, triangular, oval, T-shaped, hourglass-shaped, asymmetrical or in any other shape that appears suitable to a person skilled in the art.

In a preferred embodiment, the absorption body contains superabsorbent polymer material (SAP) to an extent of 5-100 percent by weight, preferably to an extent of 10-95 percent by weight, further preferably to an extent of 15-90 percent by weight, very particularly preferably to an extent of 20-80 percent by weight. Typically, the SAP material can absorb at least 15 times, in particular 20 times, its weight of 0.9% saline solution by weight (measured in accordance with NWSP 242.0.R2(15)).

The SAP material can, for example, be particulate or fibrous or planar or foamy.

The storage layer of the absorption body can contain further materials, such as cellulose fibers (“fluff”) or plastic fibers. It is additionally conceivable to form the storage layer of the absorption body by arranging one or more layers of different material, in particular of nonwoven material.

According to a preferred embodiment, the storage layer is substantially free of the pH regulator.

Substantially free of pH regulator means that the particular layer of the absorption body, in particular the storage layer, contains less than 10 g/m², in particular less than 7 g/m², further in particular less than 5 g/m², further in particular less than 2 g/m² pH regulator, further in particular no pH regulator. As a result, a possible functional impairment of the particular layer, in particular of the storage layer, in particular of the SAP material preferably contained in the storage layer, is advantageously avoided.

It is further preferred that the layer of the absorption body that directly faces the backsheet is substantially free of the pH regulator. This advantageously results in greater wearing comfort, in particular in the case of an incontinence article in contact with the body, since a softer haptic impression of the incontinence article is achieved from the backsheet side. It is moreover known to a person skilled in the art that particulate material can exert abrasive forces which can damage the substantially liquid-impermeable backsheet, in particular a liquid-impermeable backsheet film, of the absorbent article and can lead to leakage from the article. A layer which directly faces the backsheet and which is substantially free of pH regulator, in particular particulate pH regulator, therefore has the advantage that the risk of damaging the backsheet film in the production process or during use of the incontinence article is reduced.

Furthermore, it is found to be advantageous if the incontinence article comprises a liquid absorption layer which forms a layer of the absorption body that directly faces the topsheet. As a result, a quantity of liquid that arises during a micturition event can be rapidly absorbed, distributed, and forwarded to the at least one storage layer.

Layers for rapid absorption and distribution of bodily excretions, so-called liquid absorption or distribution layers, are already known in the technical field and typically consist of a fibrous material, in particular a nonwoven material.

The fibers can be natural or synthetic in origin and can be of a defined length (staple fibers) or continuous or be formed in situ. The fibers can be formed from a single polymer or polymer blend (single-component fiber) or from more than one single polymer and/or polymer blend (multicomponent fiber).

A multicomponent fiber has a cross-section which comprises more than one single section, each of these sections comprising a different polymer or different polymer blend. The term multicomponent fiber encompasses, but is not limited to, a bicomponent fiber. The various components of multicomponent fibers are arranged over the cross-section of the fiber in substantially different regions and extend continuously over the length of the fiber. A multicomponent fiber can have an overall cross-section which comprises subregions of the two or more different components of any shape or arrangement, including, for example, coaxial subsections, core-and-sheath subsections, side-by-side subsections, radial subsections, insular subsections, etc.

A bicomponent fiber having a “core/sheath structure” has a cross-section which comprises the following: two discrete sections, each of which consists of a polymer or polymer blend, wherein the sheath polymer or the sheath polymer-blend component is arranged around the core polymer or the core polymer-blend component.

The basis weight of nonwoven materials is generally specified in grams per square meter (g/nn²).

In a preferred embodiment, the liquid absorption layer comprises multicomponent fibers, in particular bicomponent fibers, further in particular polyester-comprising bicomponent fibers, so-called Bico/PES fibers. The multicomponent fibers, in particular the bicomponent fibers, preferably have a circular or trilobate cross-section.

Preferred combinations of components in bicomponent fibers are polyethylene terephthalate (PET)/polyethylene (PE), PET/polypropylene (PP), PET/polyester copolymers (CoPET), polylactic acid (PLA)/polylactide copolymers (COPLA), PLA/PE and PLA/PP.

As an alternative, it is known in the technical field to use a material made from chemically modified cellulose fibers, for example cross-linked cellulose fibers, as the liquid absorption layer.

The liquid absorption layer can also comprise or consist of other materials such as perforated films or foams or the like.

The liquid absorption layer can substantially completely or only partially cover the at least one storage layer of the absorption body; it can thus have substantially the same planar extent or an at least regionally smaller extent than the at least one storage layer and/or the layer of the absorption body that directly faces the backsheet in the longitudinal and/or transverse direction of the incontinence article resting flat. Preferably, the liquid absorption layer covers the at least one storage layer and/or the layer of the absorption body that directly faces the backsheet to an extent of 5-100%, in particular to an extent of 10-90%, further in particular to an extent of 15-80%, further in particular to an extent of 20-70% of the planar extent thereof.

Preferably, the liquid absorption layer is arranged at least in a region in which the micturition liquid comes into contact with the incontinence article during use. In particular, the liquid absorption layer extends across and in the region of a transverse central axis of the absorption body.

In a preferred embodiment, the pH regulator is substantially positioned between the liquid absorption layer and an absorption body layer facing the backsheet. In particular, at least 70 percent by weight, further in particular 80 percent by weight, further in particular at least 90 percent by weight, further in particular at least 95 percent by weight of the pH regulator are positioned between the liquid absorption layer and the absorption body layer facing the backsheet.

Preferably, the pH regulator does not touch the topsheet when the incontinence article is dry; in particular, at least 70 percent by weight, further in particular 80 percent by weight, further in particular at least 90 percent by weight, further in particular at least 95 percent by weight of the pH regulator are positioned between a layer of the absorption body that faces the topsheet, in particular the liquid absorption layer, and the next layer of the absorption body in the direction of the backsheet.

This advantageously results in the pH regulator being arranged on a topside of the liquid absorption layer or some other absorption body layer that faces away from the user's skin during use of the incontinence article and, in particular, not being in direct contact with the user's skin.

Alternatively, the pH regulator can be substantially positioned between a topside of the liquid absorption layer that faces the topsheet and the absorption body layer that faces the backsheet, in particular substantially positioned within the liquid absorption layer.

It is found to be advantageous if the pH on a topside of the incontinence article that faces the skin is a value of 4.8-6.5, in particular 5.0-6.2, further in particular 5.2-6.0, further in particular 5.3-5.8, further in particular 5.4-5.6.

It is preferred if the incontinence article maintains a skin-friendly pH over at least two, in particular at least three, micturition events.

In an advantageous development of the above-described embodiments, what is also encompassed in the context of the present invention is an arrangement of incontinence articles, wherein the arrangement comprises at least two, in particular at least three, further in particular at least four, further in particular at least five, further in particular at least six incontinence articles, with at least a first incontinence article and a second incontinence article of the arrangement differing in at least one feature selected from the group consisting of size, absorption capacity in accordance with ISO 11948-1 (1996), amount of pH regulator in g/m², total amount of pH regulator per incontinence article, and number of absorption body layers.

The total amount of pH regulator per incontinence article, which is particularly intended for use by adults, is preferably 0.2 to 3.0 g, further preferably 0.4 to 2.5 g, further preferably 0.6 to 2.0 g.

Incontinence articles of a preferred arrangement then differ from one another in the amount of pH regulator in g/m or in the total amount of pH regulator per incontinence article or absorption capacity in accordance with ISO 11948-1 (1996) if the first incontinence article has a respective value at least 20%, in particular at least 30%, further in particular at least 40%, further in particular at least 50%, further in particular at least 75%, further in particular at least 100% greater than the respective value of the second incontinence article.

An arrangement results from the obvious preparation of the incontinence articles belonging to the arrangement, in particular from the relation or relationship of the articles to one another. This is achieved either by presentation of the incontinence articles in a common packaging unit and/or preferably by attachment of labels to the incontinence articles and/or the packaging thereof and/or presentation of the incontinence articles in physical proximity to one another or close together in terms of purpose, indicating that they belong to an arrangement. The incontinence articles forming the arrangement preferably come from the same manufacturer.

The incontinence articles forming an arrangement preferably have the same product label, such as brand names and/or subbrand names.

An arrangement composed of incontinence articles with a first expression of a feature and incontinence articles with a second expression of a feature, for example a first size and a second size, is understood as at least one representative thereof in each case and includes a plurality of the incontinence articles with a first expression of a feature and/or of the incontinence articles with a second expression of a feature or packs and packaging units containing them.

Furthermore, what is also encompassed in the context of the present invention is an arrangement of incontinence articles, wherein the arrangement comprises at least two, in particular at least three, further in particular at least four, further in particular at least five, further in particular at least six incontinence articles, with at least a first incontinence article and a second incontinence article of the arrangement being different incontinence articles selected from the group consisting of incontinence pad, open-type incontinence diaper with closure systems, closed-type incontinence diaper, and bed pad.

Further features, details and advantages of the invention will become apparent from the accompanying claims and from the graphic depiction and following description of preferred embodiments of the invention and examples. In the drawing:

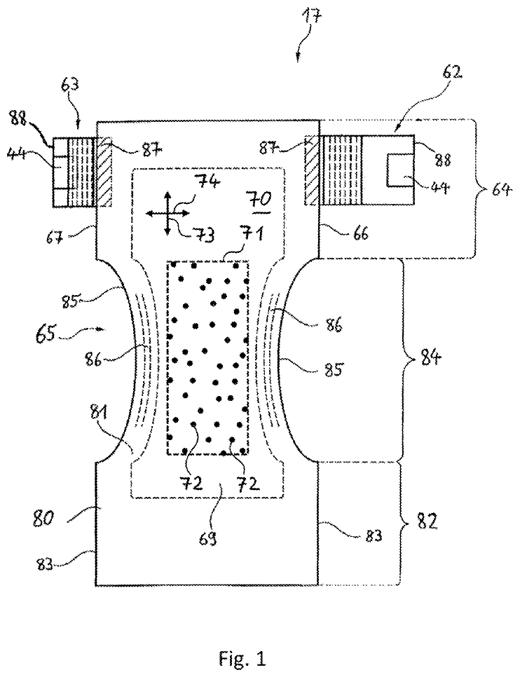

FIG. 1 shows a schematic depiction of an open-type incontinence diaper with closure elements in plan view as an exemplary embodiment of an incontinence article according to the invention

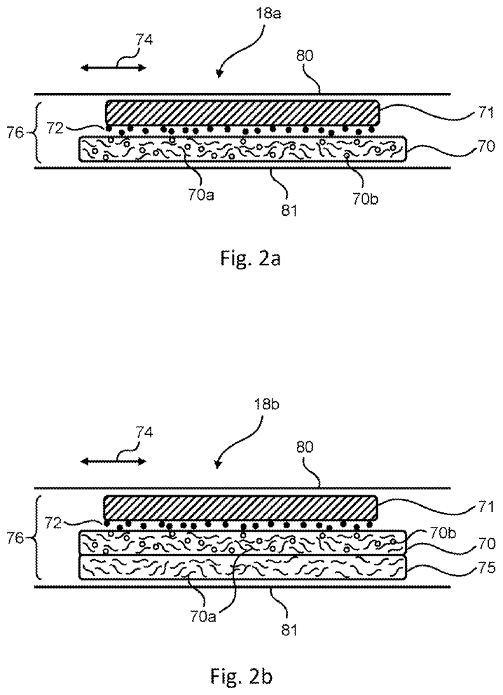

FIG. 2a shows schematically a cross-section through an incontinence article comprising a storage layer containing SAP material, a liquid absorption layer, and a pH regulator arranged in between

FIG. 2b shows schematically a cross-section through an incontinence article comprising two storage layers, SAP material, a liquid absorption layer, and a pH regulator arranged in between

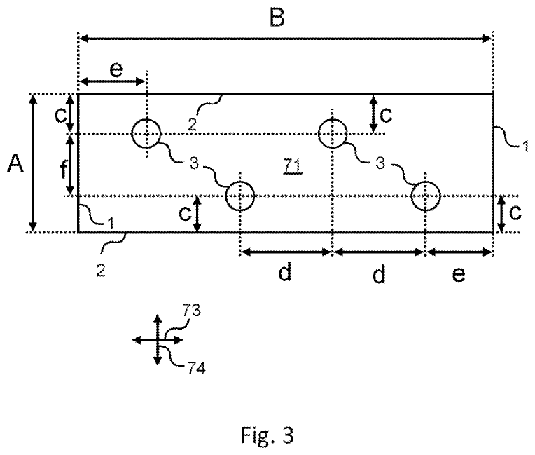

FIG. 3 shows schematically positions of measurement points for surface pH measurement relative to a liquid absorption layer

FIG. 1 shows not to scale, but schematically, an incontinence article according to the invention, denoted overall by the reference number 17, which is, by way of example, an open-type incontinence diaper with closure elements for adults in the so-called T-shape. The incontinence diaper 17 comprises a main part (chassis), denoted overall by the reference number 65, having an absorption body 69 which absorbs body fluids. In this exemplary embodiment, the absorption body 69 comprises at least one storage layer 70, containing SAP material (not shown in FIG. 1), and a liquid absorption layer 71. The incontinence diaper 17 according to the invention contains a pH regulator 72 which is substantially arranged between the liquid absorption layer 71 and the at least one storage layer 70 of the absorption body, as described in more detail in FIGS. 2a and 2b. The absorption body 69 is arranged between two planar materials, specifically an at least regionally liquid-permeable topsheet 80 and a substantially liquid-impermeable backsheet 81 of the main part of the diaper 65.

Distinguishable on the incontinence diaper 17 are a longitudinal direction 73 and a transverse direction 74 of the incontinence diaper 17, the latter direction corresponding to a direction of the hip circumference of the user when the incontinence diaper is in place. The main part 65 comprises a front region 82 having front lateral longitudinal edges 83, a back region 64 having a first rear lateral longitudinal edge 66 and a second rear lateral longitudinal edge 67, and a crotch region 84 arranged in between. Adjacent to a respective longitudinal edge 85 of the crotch region 84, the main part 65 has an elasticized section in each case, and thus an elasticized leg opening section 86. In the case depicted, these elasticized leg opening sections 86 are formed by elastic threads which run between the topsheet 80 and backsheet 81 and are fixed to the topsheet 80 and/or backsheet 81 in a pretensioned state and which are curved in an arc and are therefore oriented at least with one component in the longitudinal direction 73 of the incontinence diaper 17.

On the T-shaped incontinence diaper 17, there are provided, in the back region 64 of the main part 65 in the transverse direction 74 of the incontinence diaper 17, a first elastic diaper side part 62 which extends laterally beyond the first rear lateral longitudinal edge 66 and a second elastic diaper side part 63 which extends laterally beyond the second rear lateral longitudinal edge 67, which elastic diaper side parts are, in the region of the rear lateral longitudinal edges 66 and 67, undetachably attached in an overlap region 87 to the back region 64 of the main part 65. By contrast, no diaper side parts are provided in the front region 82.

In an alternative variant embodiment, the incontinence diaper can also be in the form of an H-shaped incontinence diaper, in which case diaper side parts are then additionally formed in the front region and preferably on both sides, as shown in WO2005102241A1 for example. As a further variant embodiment, a closed-type incontinence diaper is also conceivable, in which case there is preferably respective attachment in the front region and in the back region of a stomach part and back part, which are joined to one another at respective lateral longitudinal edges of the stomach part and back part such that the incontinence diaper is annularly closed in the direction of the hip circumference, as shown in WO2013171068A1 for example. As a further variant embodiment, the incontinence article 17 can be in the form of a bed pad or incontinence pad.

The first elastic diaper side part 62 and the second elastic diaper side part 63 of the T-shaped incontinence diaper 17 each have at least one closure element 44 in the region of their free end 88 in the transverse direction 74 of the incontinence diaper 17. The closure element 44 is in the form of a preferably rectangular tab and is folded onto itself by the manufacturer. In case of use, the closure element 44 can be opened, i.e. unfolded, in order to place the incontinence diaper 17 on a user, with the first elastic diaper side part 62 and the second elastic diaper side part 63 overlapping the front region 82 of the main part 65 and the closure elements 44 being detachably adhesively fixed on the side of the front region 82 of the main part 65 that faces away from the user.

By way of example, FIGS. 2a and 2b each show schematically, not to scale, a cross-section through an incontinence article 18a, 18b, for example an open-type incontinence diaper with closure systems (as described in more detail in FIG. 1) or a closed-type incontinence diaper or an incontinence pad or a bed pad. The incontinence articles 18a, 18b comprise a liquid-permeable topsheet 80, a substantially liquid-impermeable backsheet 81 (thus in use), and an absorption body 76 arranged in between. The absorption body 76 of the incontinence article 18a (FIG. 2a) comprises a storage layer 70 containing fibrous material 70a, for example cellulose fibers or plastic fibers, and SAP material 70b, a liquid absorption layer 71, and a pH regulator 72 arranged in between. The liquid absorption layer 71 forms a layer of the absorption body 76 that directly faces the topsheet 80.

The storage layer 70 directly faces the backsheet and is substantially free of the pH regulator 72. The absorption body of the incontinence article 18b (FIG. 2b) additionally comprises a further layer, namely a layer 75 of the absorption body 76 that directly faces the backsheet 81 and that substantially consists of fibrous material 70a, for example cellulose fibers or plastic fibers, and is arranged between the storage layer 70 and the backsheet 81. The layer 75 directly facing the backsheet 81 is substantially free of pH regulator 72.

In the exemplary embodiments depicted in FIGS. 2a and 2b, the pH regulator 72 comprises particulate monosodium citrate of the type “Fine Granular F3500” from Jungbunzlauer. Said monosodium citrate has a particle size of 80 to 355 μm to an extent of 59 percent by weight and a purity of at least 99%. The amount of pH regulator 72 in this example is 30 g/m².

The SAP material 70b is particulate in the cases depicted. In a conceivable variant, the SAP material can be fibrous or planar or foamy.

FIGS. 2a and 2b both show the cross-section through the incontinence article 18a and 18b, respectively, in the transverse direction 74, in the case of an incontinence diaper 17, for example in the crotch region (reference number 84 in FIG. 1, the elasticized leg opening sections 86 not depicted).

The liquid absorption layer 71 only partially covers the storage layer 70 of the absorption body 76 and, in the present case, has a smaller extent than the storage layer 70 (FIG. 2a) or the storage layer 70 and the layer 75 of the absorption body 76 that directly faces the backsheet 81 (FIG. 2b) in the transverse direction 74 of the incontinence article 18 resting flat. Alternatively, the liquid absorption layer can completely cover the storage layer and/or the layer 75 of the absorption body 76 that directly faces the backsheet 81.

In this example (FIG. 2b), the storage layer 70 has, in the transverse direction 74, substantially the same extent as the layer 75 of the absorption body 76 that directly faces the backsheet 81. However, the storage layer 70 and the layer 75 of the absorption body 76 that directly faces the backsheet 81 can also have different extents.

### EXAMPLE 1: PH REGULATION BY MONOSODIUM CITRATE OR DISODIUM CITRATE COMPARED TO CITRIC ACID

1A: An adult incontinence diaper was produced with a pH regulator consisting of monosodium citrate (first incontinence diaper 1A, according to the invention). The incontinence diaper 1A comprised the following components in the arrangement specified:
Topsheet: SMS nonwoven material, 12 g/m², type 3000063 from Avgol LTD
Liquid absorption layer: Nonwoven material carded and thermally bonded (“air-through-bonded”), Bico/PES, 40 g/m², dimensions of 90×270 mm (0.0243 m²), type 11040WC0A from Berry
pH regulator: 25 g/m² monosodium citrate, purity of >95%, nonhydrogenated, total amount per incontinence article of 0.6 g, calculated total molar amount of monosodium citrate per incontinence article of 0.003 mol, catalog number 21533 from Thermo Fisher Scientific
Storage layer: 21.5 g of cellulose fibers (“fluff”), type CoosAbsorb S from Resolute; mixed with 13.5 g of particulate SAP material (type SXM 9791 from Evonik)
Layer of the absorption body that faces the backsheet: 34.8 g of cellulose fibers (“fluff”), type CoosAbsorb S from Resolute
Backsheet: Nonwoven film laminate, 18 g/m², Hyfol PE soft textile, type 14202 from RKW

1B: A further adult incontinence diaper was produced with a pH regulator consisting of disodium citrate (second incontinence diaper 1B, for comparison, according to the invention).

The incontinence diaper 1B comprised the following components in the arrangement specified:
Topsheet: SMS nonwoven material, 12 g/m², type 3000063 from Avgol LTD
Liquid absorption layer: Nonwoven material carded and thermally bonded (“air-through-bonded”), Bico/PES, 40 g/m², dimensions of 90×270 mm (0.0243 m²), type 11040WC0A from Berry
pH regulator: 31 g/m² disodium citrate, purity of 99%, hydrated form (disodium hydrogencitrate sesquihydrate), total amount per incontinence article of 0.75 g, calculated total molar amount of disodium citrate per incontinence article of 0.003 mol, catalog number 25024 from Thermo Fisher Scientific
Storage layer: 21.5 g of cellulose fibers (“fluff”), type CoosAbsorb S from Resolute; mixed with 13.5 g of particulate SAP material (type SXM 9791 from Evonik)
Layer of the absorption body that faces the backsheet: 34.8 g of cellulose fibers (“fluff”), type CoosAbsorb S from Resolute
Backsheet: Nonwoven film laminate, 18 g/m², Hyfol PE soft textile, type 14202 from RKW

1C: A further adult incontinence diaper was produced with a pH regulator consisting of citric acid (third incontinence diaper 1C, for comparison, not according to the invention). The incontinence diaper 1C comprised the following components in the arrangement specified:
Topsheet: SMS nonwoven material, 12 g/m², type 3000063 from Avgol LTD
Liquid absorption layer: Nonwoven material carded and thermally bonded (“air-through-bonded”), Bico/PES, 40 g/m², dimensions of 90×270 mm (0.0243 m²), type 11040WC0A from Berry
pH regulator: 26 g/m² citric acid, purity of at least 99.5%, nonhydrogenated, total amount per incontinence article of 0.625 g, calculated total molar amount of citric acid per incontinence article of 0.003 mol, article number 471A1F, “citric acid anhydrous pharm” from Barcelonesa
Storage layer: 21.5 g of cellulose fibers (“fluff”), type CoosAbsorb S from Resolute; mixed with 13.5 g of particulate SAP material (type SXM 9791 from Evonik)
Layer of the absorption body that faces the backsheet: 34.8 g of cellulose fibers (“fluff”), type CoosAbsorb S from Resolute
Backsheet: Nonwoven film laminate, 18 g/m², Hyfol PE soft textile, type 14202 from RKW

The pH regulator was arranged between the liquid absorption layer and the storage layer in the first incontinence diaper 1A and second incontinence diaper 1B and third incontinence diaper 10.

For better comparability of pH regulation in the first incontinence diaper 1A and second incontinence diaper 1B and third incontinence diaper 10, the respective total amount of pH regulator per incontinence article was chosen such that a substantially equal total molar amount of the respective pH regulator per incontinence article of 0.003 mol was present in each case (see Table 1).

TABLE 1
pH regulators used in Example 1

| | 1A | 1B | 1C |
|---|---|---|---|
| | (with MSC) | (with DSC) | (with CA) |
| Water of crystallization | Non- | Sesqui- | Non- |
| | hydro- | hydrate | hydro- |
| | genated | | genated |
| Molarity [g/mol] | 214.11 | 263.1 | 192.13 |
| Total amount per incontinence | 0.6 | 0.75 | 0.625 |
| article [g] | | | |
| Surface area covered by pH | 0.0243 | 0.0243 | 0.0243 |
| regulator [m²] | | | |
| Amount of pH requlator [g/m²] | 25 | 31 | 26 |
| Total molar amount per | 0.003 | 0.003 | 0.003 |
| incontinence article [mol] | | | |

MSC, monosodium citrate;
DSC, disodium citrate;
CA, citric acid

As described in more detail below in the test method section, three consecutive micturition events were simulated with a urine substitute liquid and the pH values were measured on a respective outer topside of the topsheet of the first and second and third incontinence article in the region of the liquid absorption layer 71. FIG. 3 shows the positions of four measurement points 3 relative to the liquid absorption layer 71 of the first and second and third incontinence article (when viewing the topsheet). A transverse extent A of the liquid absorption layer 71 was 90 mm in each case. A longitudinal extent B of the liquid absorption layer 71 was 270 mm. The pH was measured at all four measurement points 3, and the 4 measurement values at each time were used to calculate an average value, which is shown in Table 3. Moreover, the average pH values obtained after every simulated micturition were used to calculate a mean value and an associated standard deviation, which are likewise presented in Table 3. The measurement points 3 were distributed in the longitudinal direction 73 of the respective liquid absorption layer in such a way that a distance e between a measurement point 3 that is closest in the longitudinal direction 73 to a respective transverse edge 1 of the liquid absorption layer 71 and the respective transverse edge 1 that is closest in the longitudinal direction 73 was 45 mm. A distance d between two measurement points 3 following one another in the longitudinal direction 73 was 60 mm in this example.

A distance c between a measurement point 3 that is closest in the transverse direction 74 to a respective longitudinal edge 2 of the respective liquid absorption layer 71 and the respective longitudinal edge 2 that is closest in the transverse direction 74 was 25 mm. A distance f between two measurement points 3 following one another in the transverse direction 74 was 40 mm in this example.

Respective liquid volumes of the three simulated micturitions can be gathered from Table 2.

TABLE 2
Volumes of urine substitute liquid used in Example 1

| | | Proportion of maximum |
|---|---|---|
| | Liquid volume | absorption capacity |
| | [ml] | [%] |
| First simulated micturition | 300 | 45 |
| Second simulated micturition | 150 | 22.5 |
| Third simulated micturition | 150 | 22.5 |
| Total | 600 | 90 |

The pH values measured at the various times at the described four measurement points 3 can be gathered from Table 3.

TABLE 3
pH regulation, monosodium citrate and disodium citrate compared
to citric acid, pH values assigned to individual times are
average values from four measurement values in each case

| | First simulated | | | Second simulated | | | Third simulated | | |
|---|---|---|---|---|---|---|---|---|---|
| | micturition | | | micturition | | | micturition | | |
| | 1A | 1B | 1C | 1A | 1B | | 1A | 1B | 1C |
| Time | (with | (with | (with | (with | (with | 1C (with | (with | (with | (with |
| [min]* | MSC) | DSC) | CA) | MSC) | DSC) | CA) | MSC) | DSC) | CA) |
| 1 | 5.2 | 5.0 | 4.3 | 5.4 | 5.7 | 5.4 | 5.3 | 5.7 | 5.4 |
| 3 | 5.3 | 5.3 | 4.0 | 5.4 | 5.6 | 5.3 | 5.5 | 5.7 | 5.3 |
| 5 | 5.2 | 5.3 | 4.3 | 5.5 | 5.5 | 5.3 | 5.4 | 5.7 | 5.4 |
| 10 | 5.3 | 5.1 | 4.5 | 5.5 | 5.4 | 5.3 | 5.5 | 5.8 | 5.4 |
| 15 | 5.2 | 5.3 | 4.1 | 5.4 | 5.7 | 5.4 | 5.5 | 5.7 | 5.6 |
| 20 | 5.1 | 5.3 | 4.3 | 5.5 | 5.6 | 5.4 | 5.5 | 5.6 | 5.7 |
| MV | 5.2 | 5.2 | 4.3 | 5.5 | 5.6 | 5.4 | 5.5 | 5.7 | 5.5 |
| SD | 0.1 | 0.1 | 0.2 | 0.1 | 0.1 | 0.1 | 0.1 | 0.1 | 0.1 |

MSC, monosodium citrate;
DSC, disodium citrate;
CA, citric acid
MV, mean value of the preceding average pH values in the respective column for the micturition indicated in the column heading;
SD, standard deviation
*After the respective simulated micturition

The third incontinence diaper 10 containing citric acid yielded pH values of 4.0 to 4.5 after the first simulated micturition, and was distinctly below the skin-friendly range with a mean value of pH 4.3±SD=0.2. It was only with the second and third simulated micturition two and five hours after the start of the test that there was a skin-friendly pH within the range from 5.4 to 5.5 (mean values pH 5.4±SD=0.1 and pH 5.5±SD=0.1).

The second incontinence diaper 1B containing disodium citrate already yielded skin-friendly pH values of pH 5.0 to 5.3 (mean value pH 5.2±SD=0.1) after the first simulated micturition, and increased only slightly after the second and third simulated micturition (mean values pH 5.6±SD=0.1 and pH 5.7±SD=0.1).

The first incontinence diaper 1A containing monosodium citrate likewise maintained a relatively constant skin-friendly pH of 5.1 to 5.5 over three simulated micturition events and kept the pH constant at pH 5.4 to 5.5 particularly after the second and third simulated micturition (mean values pH 5.5±SD=0.1 in each case).

In particular, it was surprising that both the first incontinence diaper 1A and the second incontinence diaper 1B had an approximately identical, skin-friendly pH after the first simulated micturition (mean value pH 5.2±SD=0.1 in each case), but a distinctly higher pH than the third incontinence diaper 10 containing citric acid (mean value pH 4.3±SD=0.2), even though all three incontinence diapers had a substantially identical total molar amount of pH regulator (0.003 mol in each case). Furthermore, it was surprising that the pH values of both the first incontinence diaper 1A and the second incontinence diaper 1B, after the second and third simulated micturition, did not rise or did not rise substantially more strongly than the pH values of the incontinence diaper 10 containing citric acid (see mean values in Table 3).

The first incontinence diaper 1A containing monosodium citrate yielded only slightly better pH regulation than the second incontinence diaper 1B containing disodium citrate over the entire period of the test, with a substantially identical total molar amount of pH regulator (0.003 mol in each case). Therefore, when using monosodium citrate, approximately 20% less pH regulator can be used by weight compared to the hydrogenated disodium citrate used in this example. Thus, besides technical advantages as described above, advantages as regards economy and logistics are additionally also yielded by the choice of pH regulator.

### EXAMPLE 2: PH REGULATION BY A PH REGULATOR CONSISTING OF CITRIC ACID AND TRISODIUM CITRATE

2A: An adult incontinence diaper was produced with a pH regulator known in the prior art (WO2012121932A1) and consisting of a mixture of trisodium citrate and citric acid, the mixing ratio being 6.2:1 (w/w) (fourth incontinence diaper 2A, for comparison, not according to the invention). The incontinence diaper 2A comprised the following components in the arrangement specified:
Topsheet: SMS nonwoven material, 12 g/m², type 3000063 from Avgol LTD
Liquid absorption layer: Nonwoven material carded and thermally bonded (“air-through-bonded”), Bico/PES, 40 g/m², dimensions of 90×270 mm (0.0243 m²), type 11040WC0A from Berry
pH regulator: 31 g/m² (total amount per incontinence article of 0.75 g) trisodium citrate:citric acid in a ratio of 6.2:1 (w/w)
Storage layer: 21.5 g of cellulose fibers (“fluff”), type CoosAbsorb S from Resolute; mixed with 13.5 g of particulate SAP material (type SXM 9791 from Evonik)
Layer of the absorption body that faces the backsheet: 34.8 g of cellulose fibers (“fluff”), type CoosAbsorb S from Resolute
Backsheet: Nonwoven film laminate, 18 g/m², Hyfol PE soft textile, type 14202 from RKW

2B: An adult incontinence diaper was produced with a pH regulator known in the prior art (WO2012121932A1) and consisting of a mixture of trisodium citrate and citric acid, the mixing ratio being 7.2:1 (w/w) (fifth incontinence diaper 2B, for comparison, not according to the invention). The incontinence diaper 2B comprised the following components in the arrangement specified:
Topsheet: SMS nonwoven material, 12 g/m², type 3000063 from Avgol LTD
Liquid absorption layer: Nonwoven material carded and thermally bonded (“air-through-bonded”), Bico/PES, 40 g/m², dimensions of 90×270 mm (0.0243 m²), type 11040WC0A from Berry
pH regulator: 31 g/m² (total amount per incontinence article of 0.75 g) trisodium citrate:citric acid in a ratio of 7.2:1 (w/w)
Storage layer: 21.5 g of cellulose fibers (“fluff”), type CoosAbsorb S from Resolute; mixed with 13.5 g of particulate SAP material (type SXM 9791 from Evonik)
Layer of the absorption body that faces the backsheet: 34.8 g of cellulose fibers (“fluff”), type CoosAbsorb S from Resolute
Backsheet: Nonwoven film laminate, 18 g/m², Hyfol PE soft textile, type 14202 from RKW

The trisodium citrate used in the fourth incontinence diaper 2A and fifth incontinence diaper 2B had a purity of >99% and was present as a dihydrate (article number 27833.363 from VWR chemicals). The citric acid was present in nonhydrogenated form and had a purity of at least 99.5% (article number 471A1F, “citric acid anhydrous pharm” from Barcelonesa).

The pH regulator was arranged between the liquid absorption layer and the storage layer in the fourth incontinence diaper 2A and fifth incontinence diaper 2B.

As described in more detail with regard to Example 1 and in the test method section, three consecutive micturition events were simulated with a urine substitute liquid and the pH values were measured on the respective outer topside of the topsheet of the fourth and fifth incontinence article in the region of the liquid absorption layer 71. FIG. 3 shows the positions of the four measurement points 3 relative to the liquid absorption layer 71 of the fourth and fifth incontinence article (when viewing the topsheet). The transverse extent A of the liquid absorption layer 71 was 90 mm in each case. The longitudinal extent B of the liquid absorption layer 71 was 270 mm. The pH was measured at all four measurement points 3, and the 4 measurement values at each time were used to calculate an average value, which is shown in Table 5. Moreover, the average pH values obtained after every simulated micturition were used to calculate a mean value and the associated standard deviation, which are likewise presented in Table 5. The measurement points 3 were distributed in the longitudinal direction 73 of the respective liquid absorption layer in such a way that a distance e between a measurement point 3 that is closest in the longitudinal direction 73 to a respective transverse edge 1 of the liquid absorption layer 71 and the respective transverse edge 1 that is closest in the longitudinal direction 73 was 45 mm. A distance d between two measurement points 3 following one another in the longitudinal direction 73 was 60 mm in this example.

A distance c between a measurement point 3 that is closest in the transverse direction 74 to a respective longitudinal edge 2 of the respective liquid absorption layer 71 and the respective longitudinal edge 2 that is closest in the transverse direction 74 was 25 mm. A distance f between two measurement points 3 following one another in the transverse direction 74 was 40 mm in this example.

The liquid volumes of the three simulated micturitions can be gathered from Table 4.

TABLE 4
Volumes of urine substitute liquid used in Example 2

| | | Proportion of maximum |
|---|---|---|
| | Liquid volume | absorption capacity |
| | [ml] | [%] |
| First simulated micturition | 300 | 45 |
| Second simulated micturition | 150 | 22.5 |
| Third simulated micturition | 150 | 22.5 |
| Total | 600 | 90 |

The pH values measured at the various times at the described measurement points 3 can be gathered from Table 5.

TABLE 5
pH regulation, pH regulator consisting of citric acid and trisodium
citrate, pH values assigned to individual times are average
values from four measurement values in each case

| | First simulated | | Second simulated | | Third simulated | |
|---|---|---|---|---|---|---|
| | micturition | | micturition | | micturition | |
| Time | 2A | 2B | 2A | 2B | 2A | 2B |
| [min]* | (6.2:1) | (7.2:1) | (6.2:1) | (7.2:1) | (6.2:1) | (7.2:1) |
| 1 | 5.7 | 5.6 | 5.8 | 5.7 | 5.9 | 5.8 |
| 3 | 5.5 | 5.7 | 5.8 | 5.8 | 5.9 | 5.9 |
| 5 | 5.7 | 5.7 | 5.8 | 5.8 | 5.8 | 5.9 |
| 10 | 5.6 | 5.6 | 5.9 | 5.8 | 5.8 | 5.8 |
| 15 | 5.6 | 5.7 | 5.9 | 5.8 | 5.9 | 5.8 |
| 20 | 5.6 | 5.7 | 5.9 | 5.8 | 5.9 | 5.8 |
| MV | 5.6 | 5.7 | 5.9 | 5.8 | 5.9 | 5.8 |
| SD | 0.1 | 0.1 | 0.1 | 0.0 | 0.1 | 0.1 |

MV, mean value of the preceding average pH values in the respective column for the micturition indicated in the column heading;
SD, standard deviation
*After the respective simulated micturition

The fourth and fifth incontinence diapers 2A and 2B containing the known pH regulator consisting of a mixture of trisodium citrate and citric acid in various mixing ratios yielded pH values of 5.5 to 5.7 after the first simulated micturition. After the second and third simulated micturition, the respective pH value in the fourth and fifth incontinence diaper rose to relatively high pH values of 5.7 to 5.9. Despite different mixing ratios of trisodium citrate and citric acid, the fourth and fifth incontinence diaper 2A and 2B showed approximately identical pH regulation, and so, in this example, neither mixing ratio offered an advantage over the other.

Looking at both Examples 1 and 2 together, it becomes apparent that the pH regulator comprising monosodium citrate of the first incontinence diaper 1A or disodium citrate of the second incontinence diaper 1B was superior to all the other incontinence diapers tested—the third incontinence diaper 10, fourth incontinence diaper 2A and fifth incontinence diaper 2B— since the pH values of the first incontinence diaper 1A and second incontinence diaper 1B were within the skin-friendly range over all three simulated micturitions; in particular, the pH values remained relatively constant within a narrow range, and further in particular, there was no substantial drop in pH values after the first micturition and no substantial rise with a total of three simulated micturitions even over more than 5 hours.

### Test Method for pH Measurement on the Surface of an Incontinence Article:

A urine substitute solution is used to carry out a pH measurement on the surface of an incontinence article, in particular on an outer topside of the topsheet of the incontinence article. The urine substitute solution is a 0.9% (w/v) solution of sodium chloride (NaCl) in distilled water, prepared in accordance with ISO 11984-1 (1996), that has been adjusted to pH 6.8 using sodium hydroxide (NaOH). Such a solution has long been known in the common general knowledge in the art and the preparation thereof is part of the routine methods in the technical field.

The incontinence article to be tested is spread out flat with the topsheet side facing upward, completely unfolded if necessary, and fixed to a base if necessary.

The test is carried out in accordance with ISO 11984-1 (1996) at a temperature of 23° C.±2° C. and a relative air humidity of 50%±5%. The incontinence article to be tested and the urine substitute liquid are preconditioned accordingly in accordance with ISO 11984-1 (1996).

In order to represent a usage situation relatively realistically and to track the pH development or the buffering effect of the pH regulator over a relatively long usage period with multiple micturition events, three micturition events over a period of five hours are simulated in the course of the test as described below. Here, the incontinence article is loaded overall with a liquid volume corresponding to approximately 90% of a maximum absorption capacity of the incontinence article.

The volumes of the first and second and third simulated micturition are therefore to be chosen such that a total volume of the first and second and third simulated micturition corresponds to approximately 90% of the maximum absorption capacity of the incontinence article. At the same time, a volume of a first simulated micturition corresponds to 45% to 50% of the maximum absorption capacity of the incontinence article. A respective volume of a second and a third simulated micturition is substantially identical and corresponds in each case to 20% to 22.5% of the maximum absorbent capacity.

If the maximum absorption capacity of an incontinence article is unknown, the maximum absorption capacity is determined in advance in accordance with ISO 11984-1 (1996) on the basis of a second comparable incontinence article, for example an incontinence article which can be classified under the same absorbency and size and product type and optionally removed from the same packaging as the incontinence article to be tested.

A volume of the urine substitute solution corresponding to 50% of the maximum absorption capacity of the incontinence article is poured onto the dry incontinence article within 30 seconds for the purpose of simulating a first micturition, specifically in such a way that the urine substitute solution hits the incontinence article in a region in which the micturation liquid would also hit the incontinence article during a micturition event during use. If this region should not be absolutely certain, the urine substitute solution should hit the absorption body centrally, in particular in the region of a transverse central axis of the absorption body. If the incontinence article comprises a liquid absorption layer or liquid distribution layer, the urine substitute solution is poured onto the incontinence article especially centrally within a region formed by the liquid absorption layer or liquid distribution layer.

Two hours after starting the first simulated micturition with the first volume of urine substitute solution, a second volume of urine substitute solution corresponding to 20% of the maximum absorbent capacity of the incontinence article is poured onto the incontinence article, as described above, in order to simulate a second micturition.

Five hours after the start of loading with the first volume of urine substitute solution, a third volume of urine substitute solution corresponding to 20% of the maximum absorbent capacity of the incontinence article is poured onto the incontinence article, as described above, in order to simulate a third micturition.

After each of the three simulated micturition events, the pH is measured on the outer topside of the topsheet of the incontinence article 1, 3, 5, 10, 15 and 20 min after the end of the respective simulated micturition. To this end, the pH is measured at, in each case, 4 measurement points which are spaced from one another in the longitudinal and/or transverse direction of the incontinence article spread out flat, and an average value of the four measured values is formed. If a fluid absorption layer or fluid distribution layer is present, the measurements should be made within the region formed by said layer. In any case, the measurement points should be arranged within a region covered by pH regulator.

The measurement points should be spaced from one another, in particular should at least not overlap with one another. It is important to ensure that the measurement points on the incontinence article are not too dry in order to obtain reliable measurement results. The positions of the 4 measurement points should preferably be retained for all measurements, but it is also possible to vary the position of one or more measurement points, for example if one measurement point should be too dry at the time of a later measurement. This may be the case, for example, for incontinence articles exhibiting particularly low rewetting, in particular 15 and/or 20 minutes after the respective simulated micturition. If at least two suitable measurement points should not be available for a measurement at a specific time, the measurement is only evaluated at whatever are the other times after the end of the respective simulated micturition. If only two or three measurement points should be available for a measurement, an average value of the two or three measured values should be formed.

Owing to the routine nature of such pH measurements in the technical field, it is easily possible for a person skilled in the art to assess whether a possible measurement point is too dry.

In principle, the measurement can be carried out with any measuring device judged by a person skilled in the art to be suitable for surface pH measurement. For example, the electrodes from HANNA Instruments, models HI14142 or HI1413, are suitable. Before starting the measurement, the measuring device is calibrated according to the manufacturer's specifications.

1. An incontinence article for absorption of bodily excretions, comprising an at least regionally liquid-permeable topsheet, a substantially liquid-impermeable backsheet, and an absorption body arranged between the topsheet and the backsheet, the incontinence article comprising a pH regulator between the topsheet and the backsheet at least regionally, wherein the pH regulator comprises monosodium citrate or disodium citrate.

2. The incontinence article as claimed in claim 1, wherein the pH regulator further comprises trisodium citrate.

3. The incontinence article as claimed in claim 1, wherein the pH regulator consists of monosodium citrate.

4. The incontinence article as claimed in claim 1, wherein the pH regulator consists of disodium citrate.

5. The incontinence article of claim 1, wherein the pH regulator is particulate.

6. The incontinence article as claimed in claim 5, wherein at least 50 percent by weight of the pH regulator has a particle size of 10 to 2000 μm.

7. The incontinence article of claim 1, wherein the amount of pH regulator is 10 to 100 g/m².

8. The incontinence article as claimed in claim 1, wherein the absorption body comprises at least one storage layer which comprises a superabsorbent polymer.

9. The incontinence article as claimed in claim 8, wherein the storage layer is substantially free of the pH regulator.

10. The incontinence article as claimed in claim 1, wherein the incontinence article further comprises a liquid absorption layer which forms a layer of the absorption body that directly faces the top sheet.

11. The incontinence article as claimed in claim 10, wherein the pH regulator is substantially positioned between the liquid absorption layer and an absorption body layer facing the backsheet.

12. The incontinence article as claimed in claim 1, wherein the incontinence article maintains a skin-friendly pH over at least two micturition events.

13. The incontinence article as claimed in claim 12, wherein the pH on a topside of the incontinence article that faces the skin is a value of 4.8-6.5.

14. An arrangement of incontinence articles as claimed in claim 1, wherein the arrangement comprises at least two incontinence articles, with at least a first incontinence article and a second incontinence article of the arrangement differing in at least one feature selected from the group consisting of size, absorption capacity in accordance with ISO11948-1 (1996), amount of pH regulator in g/m², total amount of pH regulator per incontinence article, and number of absorption body layers.

15. An arrangement of incontinence articles as claimed in claim 1, wherein the arrangement comprises at least two incontinence articles, with at least a first incontinence article and a second incontinence article of the arrangement being different incontinence articles selected from the group consisting of incontinence pad, open-type incontinence diaper with closure systems, closed-type incontinence diaper, and bed pad.

16. The incontinence article as claimed in claim 6, wherein at least 50 percent by weight of the pH regulator has a particle size of 50 to 1200 μm.

17. The incontinence article as claimed in claim 6, wherein at least 50 percent by weight of the pH regulator has a particle size of 80 to 800 μm.

18. The incontinence article as claimed in claim 7, wherein the amount of pH regulator is 20 to 80 g/m², further in particular 25 to 60 g/m².

19. The incontinence article as claimed in claim 12, wherein the incontinence article maintains a skin-friendly pH over at least three micturition events.

20. The incontinence article as claimed in claim 13, wherein the pH on a topside of the incontinence article that faces the skin is a value of 5.0-6.2.
